(19) United States
(12) Patent Application Publication
(10) Pub. No.: US 2017/0215797 A1
(43) Pub. Date: Aug. 3, 2017

(54) Multifunctional Wireless Intelligent Monitor

(71) Applicant: Eugen Cretu-Petra, Dandenong (AU)

(72) Inventor: Eugen Cretu-Petra, Dandenong (AU)

(21) Appl. No.: 12/877,115

(22) Filed: Sep. 8, 2010

(30) Foreign Application Priority Data
Jun. 30, 2008 (AU) 2008903304
Jun. 22, 2009 (AU) 2009202482

(51) Int. Cl. A61B 5/00; A61B 5/11; A61B 5/1455; A61B 5/01; A61B 5/0205

(52) U.S. Cl. CPC A61B 5/6808; A61B 5/01; A61B 5/0008; A61B 5/0205; A61B 5/14552; A61B 5/1128; A61B 5/1112; A61B 5/0022; A61B 2562/0219; A61B 5/021

(57) ABSTRACT

The disclosed wireless digital monitor, if is used in a residence for one patient, comprises a detector, a pager, a video camera VC, one or more door detectors DD and a computer PC. Said detector comprises a three axis accelerometer ACC, a sensor for body temperature Tb, a sensor for surrounding temperature Te, a temperature sensor TD for urine detection in diaper, microphone with amplifier MIC, an oximeter with pulse detection and blood pressure meter, two microcontrollers working with software control data from all sensors above and transmit it to a transmitter or a transceiver. Another two temperature sensors TD1 and TD2 for detection of urine and feces are connected to detector. If said monitor is used in a health care facility it comprises a multitude of detectors identical as ones used in a residence, a multitude of pager identical to ones used in a residence, a multitude of door detectors and video cameras, a PC connected to Internet and to all phone lines. Said detector works affixed on diaper if wetness detection in diaper is needed or on pants elastic for all other features. Said pager has a display DSP, a beeper and a vibrator. Most simple monitor contains a said detector and a said pager. Said pager receiver or transceiver receives data from said detector and displays it. In monitor who comprises detector(s), pager(s) and a PC, detector transmits data to PC through its interface I. Then PC transmits its data to said pager(s). Interface I receives data from said door detectors and said video cameras. A door detector comprises two identical units located above on each side of the door. Each unit comprises an optical motion detector MD, a microcontroller and a transceiver TR.

### BACKGROUND OF THE INVENTION

1. Background-Field of the Invention

This invention relates to a system containing:
a self-contained, reusable sensing device named here Detector which wirelessly transmits detected conditions to a device named here Receiver which can be a Pager or an interface with a computer PC and a Pager. Other devices can be contained in the system. Said detection devices are named here Door Detector and Video Camera. Said Detector is affixed on a diaper or on the pants elastic of an infant or any age patient.
Said Detector and Receiver are for monitoring of:
   Separate detection of urine and feces presence in said diaper; alarm and display of said presence;
   Urine and feces count; display of said urine and feces count;
   Diaper wearer breath motion and non motion; alarm and display of said breath non motion.
   Breath count; Setup, alarm and display of said breath count
   Diaper wearer sound and its volume level; setup, alarm and display of said sound volume level.
   Diaper wearer body temperature and fever; setup, alarm and display of said body temperature and fever.
   Diaper wearer surrounding air temperature; setup, alarm and display of said air temperature.
   Detector wearer position in bed or away of bed at any time, alarm, display.
   Diaper wearer position when he or she lies in bed; setup, alarm and display of said position.
   Stand up position of diaper wearer and when it occurs; setup, alarm and display of said stand up position.
   Diaper wearer sit down position; display of said sit down position.
   Fall of diaper wearer; alarm and display of said fall.
   Detect and display of stepping; stepping count, cadence and diaper wearer speed display.
   Diaper wearer run detection; alarm and setup.
   Distance walked or run; setup, alarm and display of said distance.
   Diaper wearer speed; display of said speed.
   Direction walked or run; Setup, alarm and display of said direction.
   Total muscular activity, when and how long it occurred; display of said muscular activity, timing and energy consumed.
   Diaper wearer movement intensity; display of said intensity; setup and alarm if said intensity and timing when it occurs.
   Diaper wearer presence in a certain perimeter with a settable alarm radius. Setup, alarm and display of said perimeter;
   Diaper wearer passing through certain doors and passing direction. Setup, alarm and timing when it occurs;
   Distance between diaper wearer and attendant pager. Display of said distance;
   Oxygen content in blood; setup, display and alarm of said oxygen content;
   Pulse detection and count, display and alarm;
   Blood pressure, display and alarm;
   Diaper wearer global position. Display and alarm.
   Diaper wearer video display on said Pager display, on PC monitor, on Internet and on mobile phone.
   Connecting to any remote computer, phone, landline or mobile, through PC and Internet connection to transmit diaper wearer condition and alarm;
   Recording and display of all parameters and detections above; their timing of beginning and ending.
Said Door Detector is a wireless sensing device with transceiver. Said Door Detector is made for:
   Detector presence sensing,
   identifying said Detector ID number,
   detection of the exact location at a certain time of each said Detector
   sensing of moving direction through door of said Detector and
   wirelessly transmitting all above said data to said Receiver.
Said Video Camera remotely transmits images to said Receiver.

All above data from said Detector, Door Detector and Video Camera are sent to said Pager or to PC interface which is connected to said PC. Said PC organizes, records timing and displays that data on said PC monitor and alerts said Pager.

2. Background-Description of the Prior Art

This invention application contains additions and improvements to my monitor invention application made in USA: US2005195085. Because of sudden death syndrome in new born as well in patient and infant attendance it is needed to know if the infant or patient breathes or moves. This is important for seniors too. It is important for medical staff, parents or attendants to know if infant or patient falls down. The cause of it can be an accident or the patient internal condition. This condition could be a stroke, a heart attack, a diabetic condition and so on. Detection of breath motion or fall is very important because they can be fatal if there is no immediate care. It is also important to know if there is wetness in diaper and what is the level of fullness of it, if body temperature exceeds a certain value and if environmental temperature is in normal comfortable range. Very important particularly for infant or medical attendance in recovery period is position of the patient or infant in bed, if he or she stands up, or lay down, how much he or she moved in bed or standing, up, how many steps did and how long, what is his or her oxygen concentration in blood, pulse and blood pressure. Another important detection is if infant or patient wonders. In that case is important for parent or attendant to know where infant or patient is at any moment, when he/she stood up and started walking or running, on what door said infant or patient passed through. If infant or patient went out it is important to know his/hers global position. It is important that all these data to be organized and recorded continually in a personal computer and, in some cases, to be transmitted remotely by Internet to any phone or computer.

There is no such monitor wireless or non wireless, which contains all functions above in one device. There is no device at all which monitors positions and different movements of a patient. There are inaccurate wetness monitors which detect wetness in diaper only once and are expensive. There are separate temperature monitors or oxygen and pulse monitors or blood pressure monitors. There is no monitor at all to alarm and record if patient does not move at all anywhere he/she is, falls, arouses, if patient seats or moves, when it happened and how much movement he/she did, if patient or infant urinates or defecates and how many times did it. There is no monitor to alarm and record about how many steps and what speed patient had. There is no monitor for patients to detect, transmit wireless and record all data above and quantity of motion, direction, speed, pace, steps number and distance walked. There is no monitoring wireless system of all data above plus door detection infant/patient went through, exact location in a building or global positioning together with all above detections.

My monitor resolves these problems. It does all functions above using said Detector, said Pager, said Camera, said PC and a multitude of said Door Detectors.

There are three preferred embodiments of my invention shown in following:

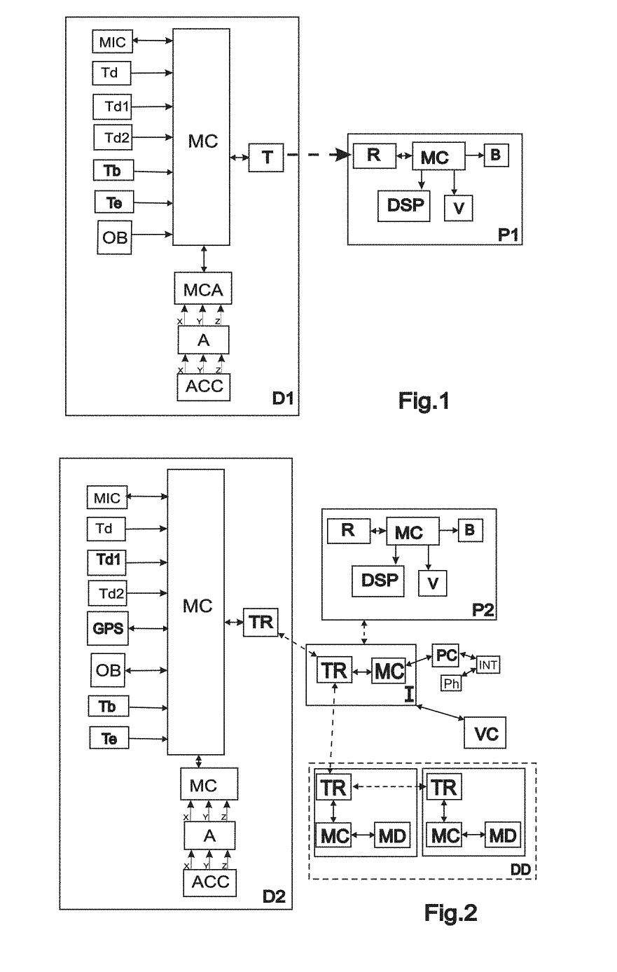

1. FIG. 1 shows a system block diagram of one of preferred embodiments of the present invention which is for the purpose of standard residential use;

2. FIG. 2 is a system block diagram of another preferred embodiment of the present invention, which is for the purpose of deluxe residential use;

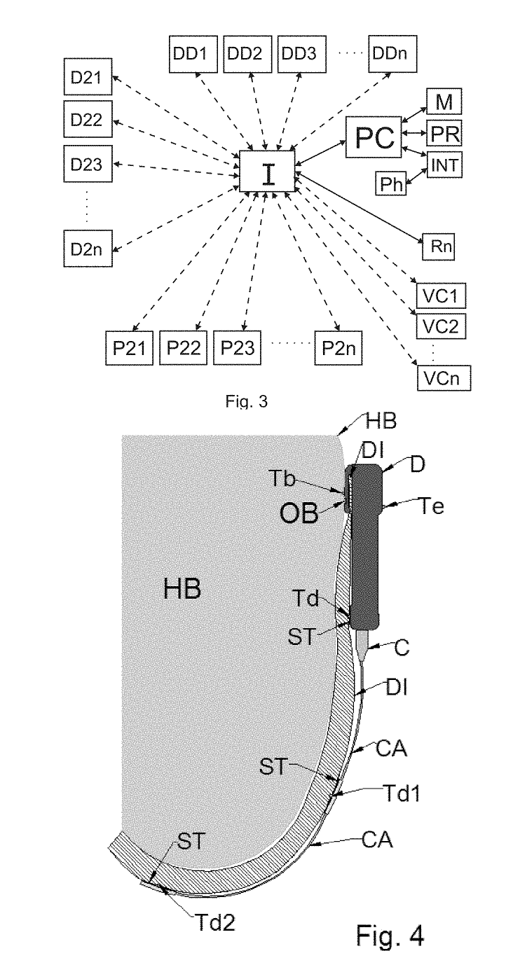

3. FIG. 3 is a system block diagram of another preferred embodiment of the present invention, which is for the purpose of healthcare institutions use.

4. FIG. 4 shows a Detector affixed on a diaper.

### DETAILED DESCRIPTION

Referring to FIG. 4, detector D in FIG. 4 can be either detector D1 or detector D2 which are correspondent to P1 and P2. Continuous lines with arrows in FIG. 1, FIG. 2, FIG. 3 and FIG. 4 show connections by solid circuits on printed circuit board or wiring inside of detectors D1 and D2 and pagers P1 and P2. Discontinuous lines with arrows show connections by radio frequency waves. Said detector D upper side is affixed on diaper DI elastic. Said detector D lower side is affixed on said diaper DI by a double side adhesive disk ST. Said adhesive disk ST has a double purpose. One purpose is to affix said detector lower side on said diaper DI. Second purpose is to ensure an intimate contact of temperature sensor Td to said diaper DI. The same affixing of said detector D lower side can be obtain by a single side adhesive tape over the detector D body and stuck on diaper DI on both sides, or by an elastic adjustable belt which goes between Detector D body and its hook and around infant or patient waist. Temperature sensor Tb stays in intimate contact with human body HB in abdominal region, being kept pressed by diaper DI or pants elastic or by said belt. One or two additional temperature sensors Td1 and Td2 can be connected through a connector C to said detector D. In FIG. 4 are shown both temperature sensors Td1 and Td2. Said sensors Td1 and Td2 are connected to said connector C through a flexible cable CA and are stuck on diaper DI with double side adhesive tapes ST in order to ensure a good contact between said sensors Td1 and Td2 and said diaper DI. FIG. 4 shows a detector DI affixed on a big size diaper DI. In this case both sensors Td1 and Td2 are needed. In this case said sensor Td2 is used as a feces sensor and Td and Td1 are used as urine sensors. If said detector D is affixed on a small size diaper DI, then only said sensor Td1 is necessary. In this case said Td1 is used as feces sensor and said Td as urine sensor. If said detector D is affixed on a newborn diaper DI or other very small diaper DI, like a cat or a small dog diaper DI, then said sensor Td is used as both urine and feces sensor. In order to sense only the bowel movement, said feces sensor Td1 or Td2 must be stuck on said diaper DI in the diaper wearer anus area. All said temperature sensors Td, Td1, Td2, Tb, Te shown in FIG. 4 in green color are injected together with discs made of elastic plastic or rubber shown in yellow color.

Said standard residential version of my invention embodiment shown in FIG. 1 contains a Detector D1 and a Pager P1. Said Detector D1 contains a microphone circuitry MIC, a diaper temperature sensor Td, a human body temperature sensor Tb, an environmental air temperature sensor Te, a diaper temperature sensor Td1, a diaper temperature sensor Td2, a microcontroller MC, a three axis low-g micro machined accelerometer ACC with an amplifier A and its microcontroller MCA, a Reflective Pulse Oximeter with a Blood pressure meter and pulse detector OB and a transmitter T. Said Pager P1 contains a receiver R a microcontroller MC, a beeper B, a vibrator V and a display DSP.

Said microphone circuitry MIC, said sensors Td, Td1, Td2, Tb, Te and said oximeter with blood pressure meter and pulse detection OB are connected to said microcontroller MC inputs.

Said deluxe residential version of my invention embodiment shown in FIG. 2 contains a Detector D2, a Pager P2, an interface I, a Computer PC with connection to any landline or mobile phone through Internet, a multitude of Door Detectors and a video camera VC with transceiver TR and a microcontroller MC. Said video camera VC communicates with said interface I. Said Detector contains a microphone circuitry MIC, a diaper temperature sensor Td, a body temperature sensor Tb, an environmental air temperature sensor Te, a microcontroller MC, one or two temperature sensors Td1 and Td2, a Three Axis Low-g micro machined Accelerometer ACC, a Reflective Pulse Oximeter with a Blood pressure meter OB and a transceiver TR. Said Pager P2 contains a transceiver TR, a microcontroller MC, a beeper B, a vibrator V and a Display DSP. Said interface contains a transceiver TR, and a microcontroller MC. Computer PC is any computer currently on the market which is able to connect to Internet and to any phone Ph, anywhere. Said Door Detector DD contains two separate embodiments. Each of said embodiments contain a transceiver TR, a microcontroller MC and a passive optical presence and motion detector MD.

Said healthcare institution version, shown in FIG. 3, contains a multitude of said Detectors D2n, a computer PC with said interface I, a multitude of said pagers P2n, a multitude of said video cameras VCn and a multitude of said door detectors DDn.

Said detectors D1, D2, said pagers P1 and P2 and said door detectors DD are individually power supplied by batteries.

Said microcontrollers MC of said Detectors D and D2n, Pagers P and P2n, Door Detectors DD, Video cameras VC, Interface I and computer PC software are working by specially created software which are copyrights and allow batteries of said components above work much longer. Same said software allows all electronics to be much smaller and to work with much less components than it would work with normal components without software.

### How it Works

In standard residential version shown in FIG. 1 microphone circuitry MIC sends signals proportional with sound volume to analog digital input of microcontroller MC. MC contain software which enable them to work as described in this patent application. Microcontroller MC measures at its analog digital inputs said temperature sensors Td, Te, Tb, Td1, Td2 and said sensor OB and ACC outputs and transform them in digital values. Microcontroller MC codifies digitally those signals and send them to transmitter T. Transmitter T transmits those digital codes in form of pulses and pauses by radio waves to pager's receiver R. Said receiver R sends those pulses to pager microcontroller MC. Pager microcontroller MC works by its software and displays the data coming from detector D on said pager P display DSP. Td, Td1, Td2, Tb and Te are all temperature sensors. Temperature sensors Td, Td1, Td2 are in intimate contact with diaper DI exterior surface by double side adhesive pads. When urination or bowel movement occurred there is an increase of temperature of 1.5 to 2 Celsius degrees at the diaper DI surface. That is because always anything comes out from a human body has more temperature than diaper exterior surface. Urination or feces liquids are immediately absorbed in diaper and that leads to an increase of diaper surface temperature. This sudden increase is interpreted by microcontroller MC as an urination or bowel movement. The difference between urination and defecation is done by position of temperature sensor where the temperature increase occurred. When an urination or bowel movement occurred said pager receives the alarm and displays that there is an urination or a bowel movement. Said pager also counts how many times these events occurred. So, no matter how much humidity is in said diaper DI at a certain moment, urination or defecation is always detected and counted.

Sensor Tb is in contact with diaper DI wearer abdominal skin. The signal it transmits to microcontroller MC is correlated to said diaper wearer body temperature. Body temperature is transmitted digitally to pager by radio waves and if it overpasses the limit setup by user in pager P1 said pager P1 beeps, vibrates and displays current alarm.

Sensor Te sends to microcontroller MC data about temperature at the said detector D exterior surface. If diaper DI wearer is uncovered or it is too cold or too hot, said pager P1 alarms the attendant. Alarms are given if measured pulse, oxygen concentration in blood and blood pressure are out of setup limits. Said three axis accelerometer ACC sends its data on three lines to its amplifier and its microcontroller MC. Each line corresponds to variation in accelerations on all three axis. Position of said accelerometer ACC is given by said Detector D1 printed circuit board contained inside of said detector D1. Ultimately position of said accelerometer ACC and its three outputs depend on said detector D1 position. Being affixed on said diaper DI, the accelerometer ACC output depends on diaper wearer position. Tests made with prototype demonstrated that even the slightest breathing motion is detected with said accelerometer ACC and its amplifier A. Different positions of diaper wearer are detected with accelerometer ACC. Fall detection is possible by a combination through software of a certain motion intensity in a certain direction. All data from accelerometer ACC are transmitted, received and displayed in real time.

In deluxe residential version shown in FIG. 2 data from microphone circuitry, sensors Td, Td1, Td2, Tb and Te, OB and ACC works identically as in standard residential version of my invention.

Said global positioning device GPS enclosed in said detector D2 sends data about said detector wearer global positioning to microcontroller MC, transceiver TR, interface with computer I, computer PC and said pager P2. All data coming from said detector D2 go first to computer PC by its interface I. Said computer PC sends data to pager P2 and memorizes, organizes and displays said data. If necessary computer PC calls any phone Ph said computer PC has been programmed to call or transmits data to other computer or computers. That way an alarm or any information can be transmitted out of residence.

Said door detector DD purpose is to detect when and if a detector D2 affixed on an infant or patient diaper or pants goes through a certain door, what passing direction said detector D2 has and in what room said Detector is. Said door detector DD communicates through radio with said interface I. Said door detector DD is composed by two separate identical units. Each unit contains an optical motion detector MD, a microcontroller MC and a transceiver TR. These units are positioned on each side of the door, on top of said door. Optical motion detectors are oriented in such a way they detect a detector D2 wearer when is very close to door. When one of optical detectors detects a detector D2 wearer approaching the door, said door detector transceiver TR transmits a call for said detector identification to said detector D2. If more than one detector D2 responds to this call said door detector transceiver TR receiver considers as door closest the diaper wearer detector whose transmission signals are strongest. If second optical detector MD is activated and the same identification code is received by second door detector transceiver TR it means that said detector D2 passed through the door. One of the door detector DD transceiver TR transmits to said computer PC by its interface I the information that a certain identified detector D2 passed through that certain door in that certain direction. Computer PC sends by its interface I this information to a certain pager P2 as it has been setup. If each door in the residence, including the exit door has a door detector DD installed, attendant would know where is the infant or patient, or if he or she got out from the house and will receive an alarm at her or his pager. Said computer records all infant or patient movements and data and calls any phone.

Said video camera VC sends images of patient to said interface I.

In a healthcare institution version of embodiment of my invention shown in FIG. 3 a multitude of said detectors D2 are shown as D21, D22, D23, . . . D2n. Said computer PC interrogates sequentially for data, at short time intervals through its said interface I, each said detector D21, D22, . . . , D2n, said retransmission relays R1, R2, . . . Rn and each door detectors DD1, DD2, . . . DDn. Data received by PC through its interface I is recorded, organized and displayed on its monitor M. Said computer PC sends any necessary data to any certain pager P2n of any certain attendant, as it was setup, about any alarm or new data about a certain patient or infant which is in said attendant responsibility and care.

All said residential and institutional versions Detectors D1 and D2 can work on diaper or on pants. Said “on diaper” or “on pants” selection is done by said detector D1 or D2 button. When “on pants” mode has been selected, sensors Td, Td1 and Td2 outputs are inhibited by said microcontroller MC software. In order to avoid unnecessary exposure to electromagnetic radio waves and unnecessary power consumption from said detectors batteries, transmissions are done at time intervals of 2 seconds to 15 minutes or anytime an alarm is necessary. Anyway the length of digital transmission is few milliseconds and their power is much lower than that of a mobile phone.

The claims defining the invention are as follows:

1. A wireless digital monitor as in FIG. 1 comprising a said Detector (D1) which communicates by radio frequency to said Pager (P1);

2. A wireless digital monitor as in FIG. 2 comprising a computer (PC) which communicates by radio through an interface (I) with said detector (D2), Pager (P2), video camera (VC) and one or a multitude of door detectors (DD).

3. A wireless digital monitor as in FIG. 3 comprising a computer (PC) which communicates by radio frequency and through its interface (I) with one or a multitude of Detectors (D2n), one or a multitude of door detectors (DDn), one or a multitude of Pagers (P2n) and one or a multitude of video cameras (VCn);

4. A wireless digital monitor as claimed in claim 1 wherein said Detector upper side being affixed to said diaper (DI) or pants elastic by its hook and by said Detector lower side being stuck on diaper (DI) by a double side adhesive disk (ST);

5. A monitor as claimed in claim 1 wherein said Detector (D1) has a temperature sensor (Td) located on said Detector lower side, said sensor being stuck by said adhesive disk (ST) and wired to microcontroller (MC);

6. A monitor as claimed in claim 1 wherein said Detector (D1) has a temperature sensor (Tb) located on said Detector (D1) hook and wired to microcontroller (MC);

7. A monitor as claimed in claim 1 wherein said Detector (D1) has a temperature sensor (Te) located on said Detector (D) opposite side to said diaper (DI) and sensor (Tb);

8. A monitor as claimed in claim 1 wherein said Detector (D) comprising inside of its body a three axis accelerometer (ACC) connected electrically to an amplifier (A) and a microcontroller (MCA) which is wired to a radio transmitter (T) or a radio transceiver (TR);

9. A monitor as claimed in claim 1 wherein said Detector (D1) comprises a Reflective Pulse Oximeter with a Blood pressure meter and pulse detector (OB) with its sensor located on Detector (D1) hook and said Reflective Pulse Oximeter with a Blood pressure meter and pulse detector (OB) is wired to microcontroller (MC);

10. A monitor as claimed in claim 1 wherein to said Detector (D1) is connected by a connector (C) two temperature sensors (TD1 and TD2), both wired to microcontroller (MC);

11. A monitor as claimed in claim 2 wherein each of said Detector (D2) comprises all sensors comprised in said Detector (D1) plus a global positioning device (GPS) wired to microcontroller (MC);

12. A monitor as claimed in claim 2 wherein said computer (PC) is connected through said computer interface (I), its monitor (M), to Internet (INT) and to telephone lines (Ph) through said Internet and communicates by radio with said Pagers (P2n);

13. A monitor as claimed in claim 2 wherein each of door detectors (DD) comprises two identical units positioned above each side of the door and each unit contains an optical motion sensor (MD) is oriented in such a way to detect a detector wearer only when is very close to door, a transceiver (TR) and a microcontroller (MC);

14. A monitor as claimed in claim 3 wherein each of said Detector (D2n) comprises all sensors comprised in detector (D2) as claim 2;

15. A monitor as claimed in claim 3 wherein each of said door detectors (DDn) are identical to door detectors (DD) in claim 2;

16. A monitor as claimed in claim 3 wherein each of said video cameras (VCn) are identical to video cameras (VC) as in claim 2;

17. A wireless digital monitor substantially as herein before described with reference to FIGS. 1-4 of the accompanying drawings.
